(19) United States
(12) Patent Application Publication
(10) Pub. No.: US 2025/0176955 A1
(43) Pub. Date: Jun. 5, 2025

(54) TISSUE OR ORGAN POSITION SYSTEM FOR INTRA-BODY USE IN A PATIENT AND METHODS OF POSITIONING TISSUES OR ORGANS WITHIN THE BODY OF A PATIENT

(71) Applicant: Boehringer Technologies, LP, Phoenixville, PA (US)

(72) Inventors: William C. DACKIS, Philadelphia, PA (US); Christopher L. Radl, Malvern, PA (US); Steven Moulden, West Chester, PA (US); Nathan Simasek, Downingtown, PA (US)

(21) Appl. No.: 18/921,299

(22) Filed: Oct. 21, 2024

Related U.S. Application Data
(60) Provisional application No. 63/606,333, filed on Dec. 5, 2023.

(51) Int. Cl. A61B 17/02; A61B 17/00

(52) U.S. Cl. CPC A61B 17/0218; A61B 2017/00477; A61B 2017/00566; A61B 2017/0212; A61B 2017/0225

(57) ABSTRACT

A system and method of use for positioning an anatomic structure at a desired position within the body of a patient. The system includes a suction assembly, a suction adapter, and a clip. The suction assembly includes a collapsible suction head, a flexible suction tube, and a retraction filament, all of which are configured for insertion into the body of the patient as a unit through a conventional trocar. The retraction filament is configured to be pulled back out of the patient's body to externalize a portion of the suction tube, whereupon a portion of it can be cut off to produce an open free end. The open free end is configured to be connected via the suction adapter to a source of suction, whereupon regulated suction is applied by the suction head to secure the anatomic structure to it and once that has occurred to elevate the anatomic structure to a desired position. The clip is applied to the externalized portion of the suction tube to hold the suction head with the anatomic structure in place at the desired position.

### FIELD OF THE INVENTION

This invention relates generally to medical devices and methods of use and more particularly to systems and methods for positioning, e.g., retracting or lifting, an anatomic structure, e.g., an organ or tissue, within the body of a patient, e.g., within the patient's abdominal cavity.

### CROSS-REFERENCE TO RELATED APPLICATIONS

This utility application claims the benefit under 35 U.S.C. § 119 (e) of Provisional Application Ser. No. 63/606,333, filed on Dec. 5, 2023, entitled “Tissue Or Organ Position System For Intra-Body Use In A Patient And Methods Of Positioning Tissues Or Organs Within The Body Of A Patient”, the entire disclosure of that provisional application is incorporated by reference herein for all purposes.

### SPECIFICATION

### Background of the Invention

During laparoscopic procedures in the abdomen an instrument, e.g., a grasper or retractor, may be used to lift or otherwise move an organ to a position so that it does not interfere with the laparoscopic procedure on another organ or anatomic structure/tissue (the “target”) within the abdomen. The organ to be moved or lifted is susceptible to being scraped, punctured, bruised, or otherwise damaged using the conventional retractors, clamps. or graspers during laparoscopic surgical procedures to move it while the surgeon works on the target. This is particularly true of the liver. Thus, some retractors include inflatable balloons and the like so to avoid unintentional damage to the organ being moved. However, such retractors are rather large and hence may block the field of view of the surgeon.

U.S. Pat. No. 11,166,710 (the “'710 patent”), which is assigned to the same assignee as the subject invention and whose disclosure is incorporated by reference herein, discloses an intra-abdominal liver retraction device and methods of use, which overcomes some of disadvantages of the prior art organ retractors. In particular, the device of the '710 patent includes a flexible sling, an anchor, and a filament. The sling has a base edge and a pair of side edges tapering toward a leading edge. The sling also includes a stiffener extending across it adjacent the base edge to enable it to be gathered and inserted through a port into the abdomen, whereupon it can be opened. The anchor is configured to be releasably secured to the diaphragm with the base edge adjacent the diaphragm so that the liver can be disposed on the sling. The filament is connected to the sling adjacent the leading edge and configured to be drawn through an aperture in the abdominal wall to lift the sling with the liver thereon upwards.

U.S. Pat. No. 11,246,580 (the “'580 patent”), which is also assigned to the same assignee as the subject invention and whose disclosure is incorporated by reference herein, discloses an intra-abdominal liver retraction device and methods of use which overcome some disadvantages of prior art organ retractors. One device of the '580 patent includes a body, a stabilizing member, and a lifting filament. The stabilizing member is configured to be swiveled so that the device can be introduced through a trocar into the abdomen of a patient. The filament pulls the stabilizing member into engagement with the abdominal wall while a foot section lifts the liver. Another device of the '580 patent includes first, second and third sections coupled together by a filament. The first and second sections are pivotably connected by a first pivotable joint. The second and third sections are pivotably connected by a second pivotable joint including the flexible filament. The second and third sections are pivoted with respect to each other to form a support surface to lift the liver by pulling the filament. The second pivotable joint separates to enable the device to be removed.

For some applications, it may be desirable to make use of suction rather than mechanical lifting means, such as disclosed in the '710 and '580 patents to lift the organ or tissue.

Livac Pty Ltd provides an organ and tissue retractor, under the trademark LiVac®, for use in laparoscopic, robotic and/or open surgical procedures to elevate organs and tissue to provide access and visualization of surgical sites.

Livac Pty Ltd. is the assignee of U.S. Pat. No. 8,814,788 (Gan), which discloses a suction retractor comprising a flexible continuous dam which forms a closed loop of any shape, and which defines one or more inlet opening into an interior of the closed loop, so that suction can be applied into an interior of the continuous dam. The suction retractor may further comprise a suction tube defining a suction channel, the suction tube attached to or attachable to the continuous dam and when attached the suction channel continuous with the one or more inlet. The one or more inlets may open to a continuous channel extending throughout an interior of the closed loop. Preferably, the continuous dam is planar or substantially planar and comprises a first compacted configuration for insertion and a second open configuration for retraction. The suction retractor can be used in single incision and convention laparoscopic surgery.

While the LiVac® device may be generally suitable for its intended purposes, it may leave something to be desired from various standpoints. For example, the LiVac® device's use requires a relatively high vacuum pressure, e.g., 300 to 600 mmHg, which may present an increased risk of barotrauma to the target tissue. Moreover, the LiVac® device is reliant on a clear, flat surface directly in line with the direction of retraction (normally above the organ). This may be unavailable, and if so, may render the LiVac® device unsuitable for some anatomic orientations. Further still, the LiVac® device includes a suction tube which must be clamped to prevent loss of operating space during placement. In addition, the LiVac® device is unsuitable for deployment via a trocar. Rather it must be deployed in a wound created by means of a 12-15 mm Hg port. In effect, a trocar must be placed, removed, and then replaced into the patient to get the internal portion, i.e., organ retraction ring, of the LiVac® device in place. This action may disrupt the flow of surgery and may create opportunity for a leaky seal between the trocar and abdominal wall.

Thus, a need exists for a device which can be used during laparoscopic surgery to support a body organ or other tissue without injuring it, yet which is simple in construction, low in cost, easy to use, effective, and does not appreciably decrease the area of the surgical site and the field of view of the surgeon. The subject invention addresses that need.

### BRIEF SUMMARY OF THE INVENTION

One aspect of this invention is a system for positioning an anatomic structure at a desired position within the body of a patient. The system comprises a suction assembly, a suction adapter, and a clip. The suction assembly comprises a collapsible suction head, a flexible suction tube, and a retraction filament. The collapsible suction head is a hollow member having an internal cavity in which a foam insert is located and a peripheral edge extending about the periphery of the cavity. The collapsible suction head includes a suction port. The flexible suction tube has a first end including a first opening coupled to and in fluid communication with the suction port, a second end including a second opening, and a passageway extending between the first and second openings. The retraction filament is an elongated flexible member having a first end and a second end. The first end is anchored to the flexible suction tube adjacent the second opening. The suction assembly is configured to be introduced through a trocar or other port into the body of the patient, whereupon the collapsible suction head is located within the body of the patient, with a portion of the flexible suction tube and a portion of the retraction filament extending out of the body of the patient through the trocar or other port. The suction adapter is configured to be connected to the passageway of the flexible suction tube and to a source of suction. The clip is configured to be releasably secured to a portion of the flexible suction tube located outside the body of the patient to hold the suction assembly and the anatomic structure at a desired position within the body of the patient.

In accordance with one preferred aspect of the system of this invention the retraction filament is configured to be retracted to pull the second end of the flexible suction tube outside of the body of the patient, whereupon a portion of the flexible suction tube contiguous with the second end of the flexible suction tube can be removed to form a free end of the flexible suction tube and disconnecting the retraction filament from the flexible suction tube.

In accordance with another preferred aspect of the system of this invention the suction assembly is configured so that the peripheral edge of the collapsible hollow head can be brought into engagement with the anatomic structure within the body of the patient.

In accordance with another preferred aspect of the system of this invention the suction adapter is configured to be connected to the passageway of the flexible suction at the free end of the flexible suction tube.

In accordance with another preferred aspect of the system of this invention when the suction adapter is connected to the passageway suction from the source of suction is applied to the interior of the internal cavity and through the foam insert to cause the anatomic structure to be releasably secured by suction to the suction head.

In accordance with another preferred aspect of the system of this invention the first end of the retraction filament is anchored to the flexible suction tube within the passageway adjacent the second opening.

In accordance with another preferred aspect of the system of this invention the flexible suction tube has an outside diameter of a first dimension, and wherein the clip comprises a slot having a width which is slightly less than the first dimension.

In accordance with another preferred aspect of the system of this invention the system additionally comprises a suction regulator configured to be connected between the suction adapter and the source of suction for regulating the level of suction provided by the source of suction.

In accordance with another preferred aspect of the system of this invention the suction regulator comprises a first chamber, a second chamber, a movable diaphragm, and a biasing member. The first chamber is configured to have suction applied thereto from the source of suction. The second chamber is at atmospheric pressure. The movable diaphragm is disposed within the suction regulator separating the first chamber from the second chamber, whereupon a differential pressure exists between the first and second chambers. The differential pressure imparts a differential pressure force on the movable diaphragm. The biasing member is configured to impart a counter force on the movable diaphragm that opposes the differential pressure force.

In accordance with another preferred aspect of the system of this invention the first chamber comprises a valve seat and a movable sealing member coupled to the movable diaphragm. The valve seat surrounds an opening for fluid within the first chamber to flow therethrough. The sealing member blocks the opening when the differential pressure force exceeds the counter force imparted by the biasing member.

In accordance with another preferred aspect of the system of this invention the suction adapter is configured to be connected to a section of flexible tubing connected to the source of suction and wherein the source of suction is regulated.

In accordance with another preferred aspect of the system of this invention the foam insert comprises an open cell foam.

In accordance with another preferred aspect of the system of this invention the collapsible suction head comprises a top portion and a downwardly projecting peripheral skirt. The downwardly projecting peripheral skirt terminates in the peripheral edge. The suction port is in the top portion of the collapsible suction head.

In accordance with another preferred aspect of the system of this invention the top portion of the collapsible suction head includes a recess configured for receipt of a portion of the flexible suction tube.

In accordance with another preferred aspect of the system of this invention the flexible suction tube includes a stretchable coil section located closely adjacent the collapsible suction head.

Another aspect of this invention is a method of positioning an anatomic structure at a desired position within the body of a patient. The method comprises providing a suction assembly comprising a collapsible suction head, a flexible suction tube, and a retraction filament. The collapsible suction head is a hollow member having an internal cavity in which a foam insert is located and a peripheral edge extending about the periphery of the cavity. The collapsible suction head includes a suction port. The flexible suction tube has a first end including a first opening in fluid communication with the suction port, a second end including a second opening, and a passageway extending between the first and second openings. The retraction filament has a first end and a second end. The first end of the retraction filament is anchored to the flexible suction tube adjacent the second opening. The suction assembly is introduced through a trocar or other port into the body of the patient, whereupon the collapsible suction head is located within the body of the patient, with a portion of the flexible suction tube and the retraction filament extending out of the body of the patient through the trocar or other port. The retraction filament is retracted through the trocar or other port from outside the body of the patient to pull the proximal end of the flexible suction tube outside of the body of the patient through an exit port in the body of the patient. A section of the flexible suction tube contiguous with the second end of the flexible suction tube outside the body of the patient is removed to form a free end of the flexible suction tube and disconnecting the retraction filament from the flexible suction tube. A suction adapter is connected to the free end of the flexible suction tube and to a source of suction. The peripheral edge of the collapsible suction head is brought into engagement with the anatomic structure within the body of the patient. Suction at a predetermined value is applied to the interior of the internal cavity and through the foam insert to cause the anatomic structure to be releasably secured by suction to the collapsible suction head.

In accordance with one preferred aspect of the method of this invention the method additionally comprises positioning the suction assembly with the anatomic structure releasably secured thereto at a desired location within the body of the patient.

In accordance with another preferred aspect of the method of this invention the method additionally comprises retracting the flexible suction tube to remove any slack in the flexible suction tube between the collapsible head and an exterior surface of the body of the patient at the exit port, and releasably securing a clip to a portion of the flexible suction tube to outside the body of the patient contiguous with the exit port.

In accordance with another preferred aspect of the method of this invention the anatomic structure is an organ located within the abdomen of the patient.

In accordance with another preferred aspect of the method of this invention the organ is a liver.

In accordance with another preferred aspect of the method of this invention the predetermined value is within the range of approximately 150 to 300 mmHg.

### BRIEF DESCRIPTION OF SEVERAL VIEWS OF THE DRAWINGS

FIG. 1 is an exploded isometric view of one exemplary preferred embodiment of a system of the subject invention showing two embodiments of a clip forming a portion of the system.

FIG. 2A is an isometric view of the system of FIG. 1 configured for connection to a line/wall suction port.

FIG. 2B is an isometric view of the system of FIG. 1 configured for connection to standard suction tubing.

FIG. 3 is an enlarged isometric view of a suction cup assembly forming a portion of the system of FIG. 1.

FIG. 4 is an enlarged sectional view taken along line 4-4 of FIG. 2B.

FIG. 5 is an enlarged isometric view showing the undersurface of the suction cup assembly of FIG. 3 showing the assembly's foam insert.

FIG. 6 is an enlarged isometric view of the foam insert shown in FIG. 5.

FIG. 7 is an enlarged isometric view of suction tubing and an associated retraction filament forming a portion of the system of FIG. 1.

FIG. 8 is a sectional view taken along line 8-8 of FIG. 7.

FIG. 9 is an enlarged isometric view of a coil component forming a portion of the system shown in FIGS. 1 and 2A.

FIG. 10A is an enlarged isometric view of a suction adapter forming a portion of the system shown in FIGS. 1 and 2A.

FIG. 10B is another enlarged isometric view of the suction adapter shown in FIG. 10A.

FIG. 11 is a longitudinal sectional view of the suction adapter shown in FIGS. 10A and 10B.

FIG. 12 is a top plan view of the suction regulator of the system shown in FIGS. 1 and 2A.

FIG. 13 is side elevational view of the suction regulator shown in FIG. 12.

FIG. 14 is a longitudinal sectional view taken along line 14-14 of FIG. 12.

FIG. 15 is an enlarged isometric view of the suction adapter shown in FIGS. 1 and 2B.

FIG. 16 is another enlarged isometric view of the suction adapted shown in FIG. 15.

FIG. 17 is an enlarged longitudinal sectional view taken along line 17-17 of FIG. 15.

FIGS. 18A-18L are respective illustrations of a sequence of steps in the use of the system shown in FIG. 1 for elevating an anatomic structure, e.g., the liver of an adult human patient, within the abdomen of the patient.

### DETAILED DESCRIPTION OF THE PREFERRED EMBODIMENTS OF THE INVENTION

In the following detailed description, reference is made to the accompanying drawings which form a part hereof, and in which are shown by way of illustration embodiments that may be practiced. It is to be understood that other embodiments may be utilized, and structural or logical changes may be made without departing from the scope. Therefore, the following detailed description is not to be taken in a limiting sense, and the scope of embodiments is defined by the appended claims and their equivalents. Various operations may be described as multiple discrete operations in turn, in a manner that may be helpful in understanding embodiments; however, the order of description should not be construed to imply that these operations are order dependent. The description may use perspective-based descriptions such as up/down, back/front, left/right, and top/bottom. Such descriptions are merely used to facilitate the discussion and are not intended to restrict the application of disclosed embodiments. The terms “coupled” and “connected,” along with their derivatives, may be used. These terms are not intended as synonyms for each other. Rather, embodiments “connected” may be used to indicate that two or more elements are in direct physical contact with each other. “Coupled” may mean that two or more elements are in direct physical contact. However, “coupled” may also mean that two or more elements are not in direct contact with each other, yet still cooperate or interact with each other. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. The description may use the terms “embodiment” or “embodiments,” which may each refer to one or more of the same or different embodiments. Furthermore, the terms “comprising,” “including,” “having,” and the like, as used with respect to embodiments, are synonymous.

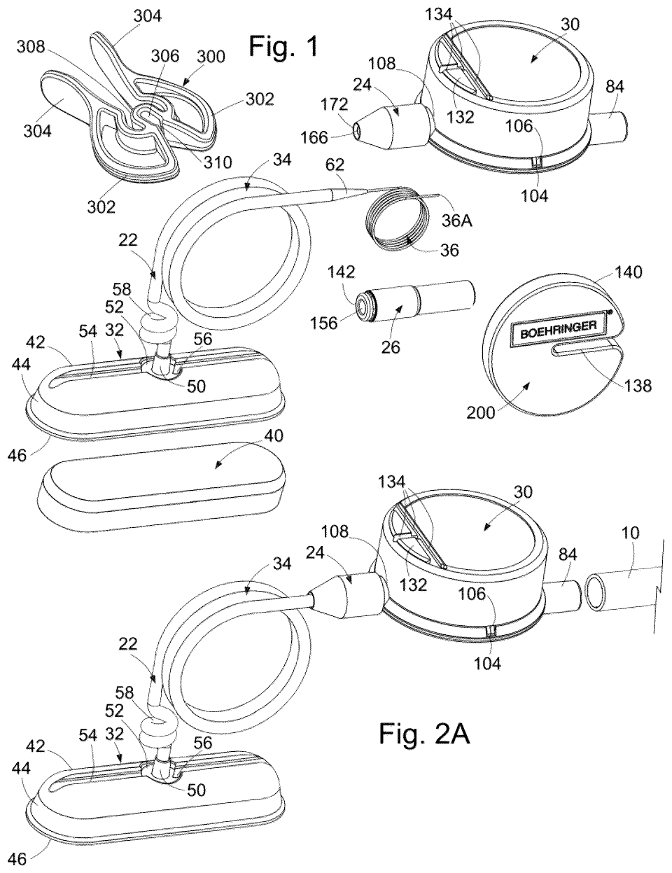

Referring now to the various figures of the drawing wherein like reference characters refer to like parts, there is shown in FIG. 1 one exemplary embodiment of an anatomic structure positioning system 20 constructed in accordance with this invention for positioning an anatomic structure e.g., tissue or an organ (hereinafter referred to as the “target tissue/organ), at a desired location within the body of a patient. In the exemplary embodiments of the invention shown, the target tissue/organ constitutes a human patient's liver 2, which is within the patient's abdominal cavity. The system 20 is configured to provide retraction of the target tissue/organ without the need for high-profile, external structures, such as bed mounts, clamps, etc. that may impede operation around the exterior of the patient during laparoscopic or robotic surgery.

The system 20 uses regulated suction to attach to the target organ/tissue 2 and maintain retraction throughout the duration of the procedure at a safe pressure for the target organ/tissue. The system includes a suction assembly 22 that is inserted into the operating field, e.g., the abdominal cavity 4 via a standard port (e.g., a conventional surgical trocar 6) and is then partially passed back through the abdominal wall 8 to be connected to a source of suction. From this placement, the suction assembly can be elevated or lowered to the desired height by manipulating the externalized portion of the suction assembly and locked into place via a clip component of the system 20 that rests externally against the abdominal wall. The system can be easily adjusted in both retraction height and tissue location without deactivating suction or removing the system from the body.

In addition to the suction assembly 22, system 20 comprises two suction adapters 24 and 26, and two embodiments of the heretofore mentioned clip (now designated by the reference numbers 200 and 300, respectively), and a suction regulator 30, all of which will be described in detail later.

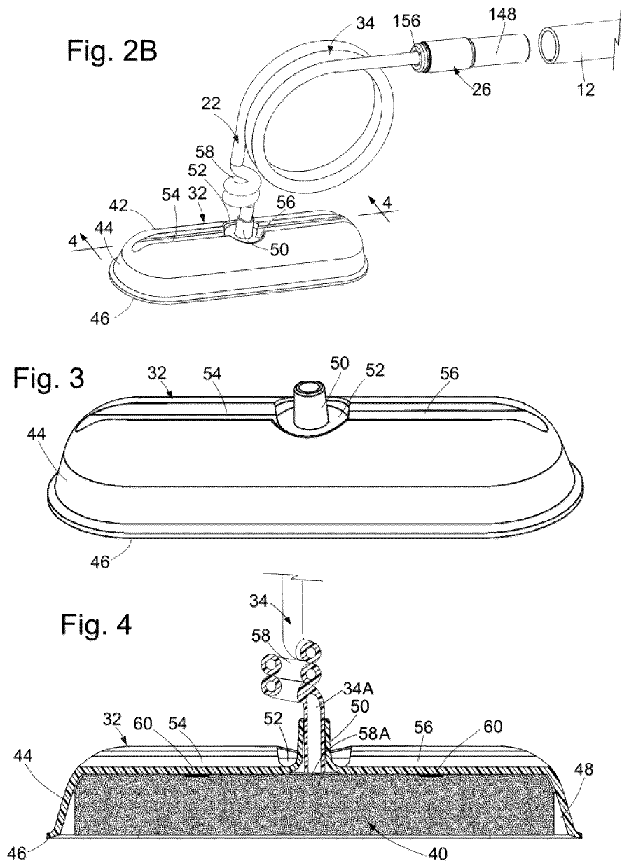

Turning first to FIG. 1, the suction assembly 22 basically comprises an assembly of a suction head 32, a suction tube 34, and a retraction filament 36. The suction tube is a conventional section of flexible tubing formed of any suitable material, e.g., polyurethane, having a central passageway 34A extending the length of the tube 34. The distal end portion of the suction tube 34 is in the form of a helical coil 58. The system 20 is configured for two types of applications. In one application, such as shown in FIG. 2A, the system 20 includes the heretofore identified suction regulator 30, which is configured to be interposed between a length of conventional flexible tubing 10 connected to a line/wall suction port (not shown) of a hospital providing suction at a first value and the suction adapter 24. The suction regulator 30 regulates the level of suction provided from the source of suction to a lesser regulated value which is provided to the suction assembly 22. In the other application, such as shown in FIG. 2B, the suction adapter 26 is interposed between a section of conventional flexible tubing 12 connected a conventional source of regulated suction (not shown) and the suction assembly 22, whereupon the regulated suction from the conventional source of regulated suction is applied to the suction assembly.

The suction head 32 is best seen in FIGS. 1-6 and basically comprises an elongated cup-shaped member which is configured to be deployed with the suction tube 34 and the retraction filament 36 into the body, e.g., the abdomen 4, of the patient through a conventional trocar 6. The trocar is introduced through the abdominal wall in the vicinity of the target organ/tissue. The suction head 32 is constructed so that it is collapsible to facilitate its deployment through the trocar and once deployed to automatically resume its normal un-collapsed state.

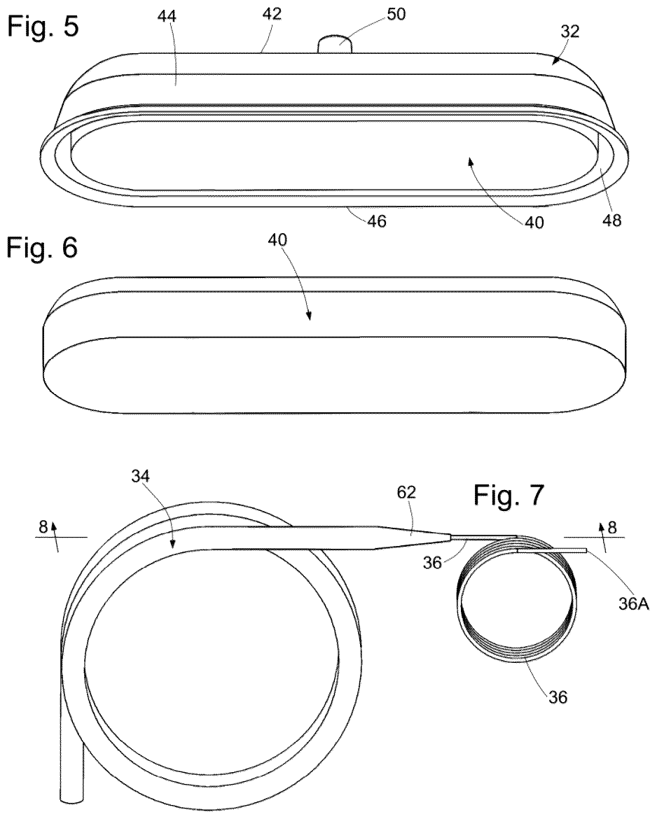

As best seen in FIGS. 4 and 5, the suction head 32 comprises an assembly of a hollow cup-shaped shell 38 and a foam insert 40. The cup-shaped shell is an elongated, e.g., oblong, member, formed of a suitable, biocompatible plastic. It includes a top wall portion 42 from which a downwardly projecting wall or skirt 44 extends. The lowermost or free edge of the skirt 44 is in the form of a peripheral flange 46. The top wall portion and the skirt together define a hollow interior space or cavity 48 in which the foam insert 40 is secured. The top wall 42 includes a centrally located upstanding nipple 50 and a surrounding annular recess 52. The nipple is configured to receive the distal end 58A of the coil 58. The coil 58 itself is best seen in FIG. 9 and as mentioned above basically forms the distal end of the suction tube 34, with the central passageway 34A of the suction tube extending through the length of the coil to the distal end 58A of the coil. The coil 58 has a plurality of helical loops enabling the coil to be stretched longitudinally. The coil is provided to account for movement/jostling/or direct contact to the cup during use. In particular, the coil acts as a buffer to reduce peak forces through the device during use. It also serves as a visual aid for users, so they don't over-tension the device during placement.

A pair of linear recesses 54 and 56 project outward diametrically from the circular recess in the top wall of the suction head. The linear recesses 54 and 56 are colinear. Each of the linear recesses is configured to receive a section of the suction tube 34 contiguous with the nipple 50 so that the contiguous portion of the suction tube, i.e., the coil 58, can be stretched to render it linear and once linearized located within that recess to reduce the cross-sectional area of the suction head and the suction tube during their deployment through the trocar into the body of the patient as will be described later.

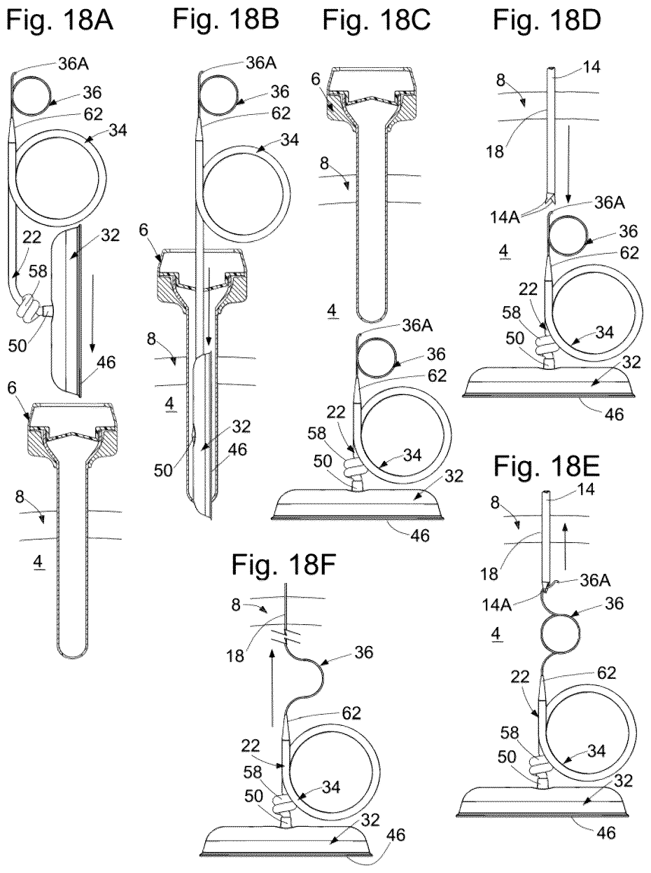

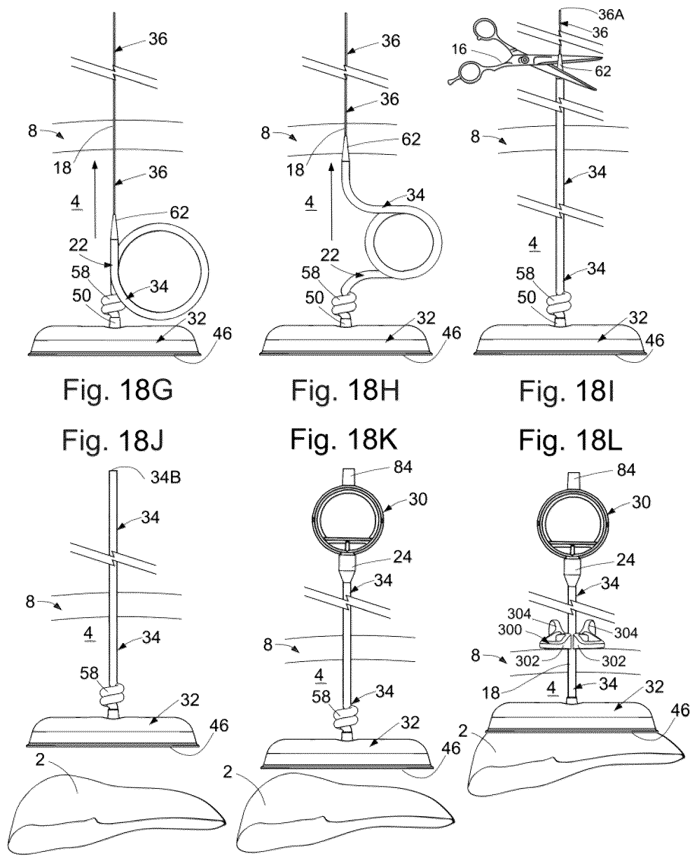

In accordance with one exemplary embodiment of a suction head 32 for positioning an organ, such as the liver of an adult human, the cup-shaped shell 38 has a length of approximately 3 inches, a width of approximately linch, a height of approximately 0.5 inch and a wall thickness of approximately 0.039 inches. That size suction head can be collapsed/folded so that it will pass through a conventional 12 mm trocar 6, like shown in FIG. 18B. The plastic material making up the cup-shaped head is preferably molded of urethane of medium hardness, e.g., preferably within the range of 70-90 Shore A durometer, and most preferably approximately 80 Shore A durometer. The relative hardness of the material making up the cup-shaped shell of the suction head provides reduced friction between it and the interior of the passageway of the trocar when the suction head is collapsed to pass through the trocar during deployment.

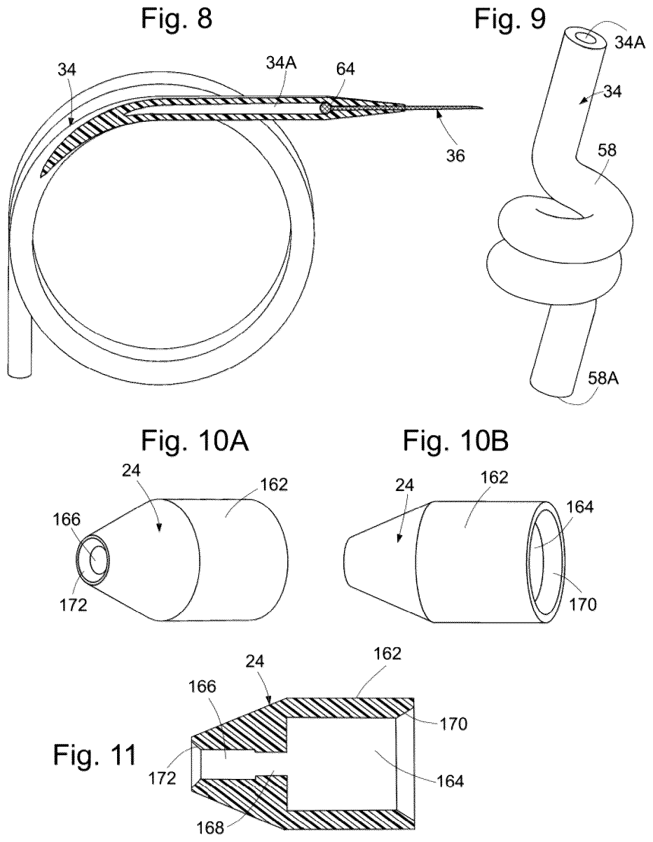

As best seen in FIGS. 4 and 8 the suction tube 34 is an elongated tubular member formed of a flexible biocompatible material, e.g., Polyurethane, and having a central passageway 34A extending therethrough. As mentioned above the suction tube is secured to the nipple 50 of the suction head. In particular, the distal end 58A of the coil 58, which is open, is fixedly secured within the nipple 50 so that the central passageway 34A extending through the suction tube is in fluid communication with the interior of the suction head's cavity 48.

In one exemplary embodiment of a system of this invention, the suction tube is approximately 18 inches in length, with an outside diameter of approximately 0.125 inch and an inside diameter of approximately 0.0625 inch. The exemplary 18 inches length of the suction tube is sufficient to reach target tissue/organ within the abdomen of a human adult patient yet provides enough remaining slack for adjustment. The inside diameter of the suction tube, i.e., the outside diameter of the central passageway 34A, restricts the allowable flow therethrough to prevent loss of pneumoperitoneum in the abdominal cavity. The outside diameter of the suction tube is small enough that the wound created by its passage through the abdominal wall does not require closure. Since the suction tube 34 is configured to be inserted with the suction head into the patient through the trocar 6 the plastic material, e.g., the Polyurethane, making up the suction tube is also preferably of medium hardness, e.g., within the range of 70-90 Shore A durometer, and most preferably approximately 90 Shore A durometer, to exhibit reduced friction.

The foam insert 40 is a flat, oblong piece of large, open-cell biocompatible, e.g., Polyester, foam, and is located within the cavity 38 of the suction head. It is fixedly secured within the cavity by a pair of welded joints 60 at the undersurface of the top wall portion of the suction head. The foam insert 40 is sized and shaped to substantially fill the cavity. This feature maintains separation between the suction tube opening 58 inside the suction head and the surface of the target tissue/organ when the suction head is releasably secured to the target tissue/organ to maintain a low-pressure cavity with sufficient suction force to retract the target tissue/organ during its positioning. The foam also provides traction and prevents the suction head from slipping along the surface of the target tissue/organ once engaged. Moreover, the open-cell foam of the insert 40 renders it suitable for compression so that it does not interfere with the collapsing of the suction head when the suction head is introduced into the patient's body through the trocar.

The annular recess 52, which surrounds the nipple 50 facilitates bending of the nipple so that the contiguous section of the flexible suction tube can be readily located within either of the linear recesses during deployment through the trocar as will be described later. Moreover, the bendability of the nipple in any direction also both prevents loss of suction due to kinking of the suction tube and allows the suction head to move relatively independently from the suction tube after it has been deployed in the body of the patient. The flexibility of this joint also makes the suction head anatomy-agnostic, such that its function is not reliant on an optimal anatomical arrangement of the target organ and surrounding landmarks.

As best seen in FIG. 8 the end of the suction tube 34 opposite the end 58 is in the form of a tapering or conical tip 62 which is heat-formed. A knotted end 64 of the retraction filament 36 is fixedly secured within the conical tip at the end of the central passageway 34A, with the remainder of the retraction filament, e.g., a length of approximately 12 inches, extending away from the conical tip. The opposite end 36A of the retraction filament is free. The retraction filament is preferably formed of braided Polyester (PET), but can be of other strong flexible, biocompatible materials, and can be a single filament or multi-filament. The tip forming process secures or anchors the retraction filament in the conical end of the suction tube. The conical shape of the heat-formed tip facilitates passage of the suction tube from the interior of the abdomen through the abdominal wall as will be described later.

As will also be described in detail later during use of the system 20 after the suction assembly 22 has been deployed into the body of the patient the retraction filament is used to pull a portion of the suction tube through the abdominal wall so that a portion of the suction tube is located outside the body of the patient, i.e., is externalized. Once that has been accomplished the conical tip is cut off to form an open free end of the suction tube and disconnect the retraction filament from the suction tube. The open free end of the suction tube can then be connected to one of the suction adapters 24 or 26 to provide regulated suction to the suction head. As will be described in detail later regulated suction may be provided to the suction head via the suction adapter 24 from the suction regulator 30 as shown in FIG. 2A. Regulated suction can also be applied to the suction head from another source of regulated suction (e.g., a conventional suction regulator —not shown) via the suction adapter 26 as shown in FIG. 2B.

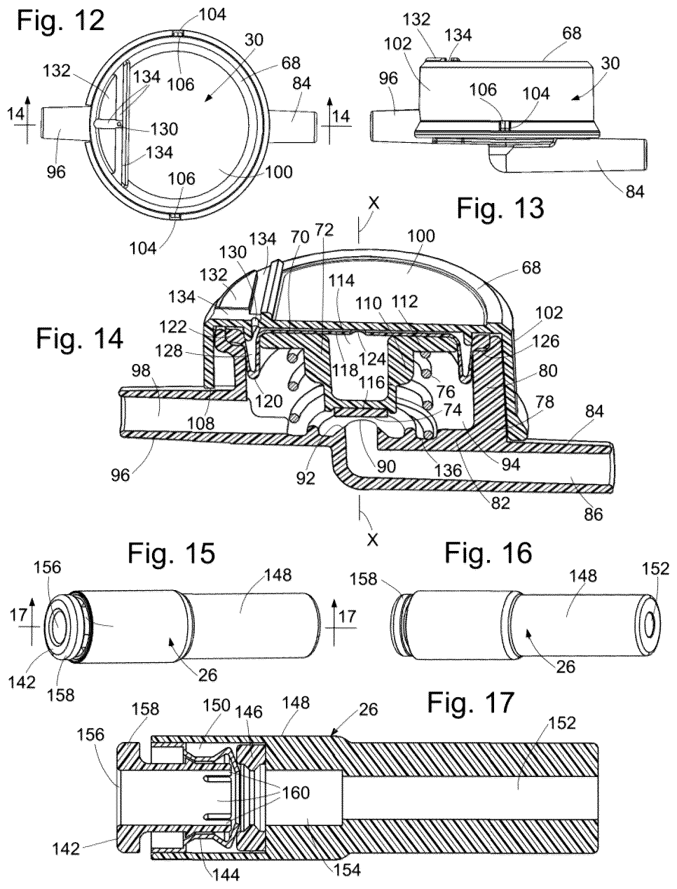

Turning now to FIGS. 12-14, the details of the suction regulator 30 forming a portion of the system 20 will now be described. That suction regulator is preferably constructed in accordance with teachings of U.S. Pat. No. 11,389,318, which is assigned to the same assignee as the subject invention, and whose disclosure is incorporated herein by reference for all purposes.

The suction regulator 30 is configured to regulate the amount of suction from a first value provided by the source of suction, e.g., a hospital's wall suction line, to a regulated value that is lower than the first value and to apply that regulated suction to the suction assembly 22. The regulated suction provided by the suction regulator 30 for positioning an organ such as the liver of a patient is in the range of approximately 150-300 mmHg and preferably approximately 200 mmHg.

The suction regulator basically comprises a lid or cover 68, a flexible diaphragm 70, a piston 72, a sealing disk 74, a helical compression spring 76, and a housing body 78. The lid or cover and the housing body are configured to be connected to form a hollow housing assembly for housing the other components making up the regulator 30. The housing body 78 and the cover 68 are each formed of a rigid plastic, such as ABS. The housing body 78 includes a circular annular sidewall 80 projecting upward from a bottom wall 82. The sidewall 80 extends about a central axis X. A tubular extension 84 projects outward radially from the central axis X. The extension 84 includes a linear passageway section 86 whose outer or free end is open. The open free end of the extension 84 forms what can be referred to as the line suction port. The port 84 is configured to be connected to a source of suction, e.g., a hospital's wall/line suction port, via the flexible tubing 10. The opposite end of the linear passageway section 86 terminates in an axially directed passageway section 88 centered about the axis X and terminates at the bottom wall 82 of the housing. The upper end of the passageway section 88 is open at 90. The portion of the bottom wall contiguous with the opening 90 forms a beveled or conical surface valve seat 92. The opening 90 is in fluid communication with a lower chamber 94 within the interior of the suction regulator 30. The lower chamber 94 is partially defined by the inner surface of the annular sidewall 80 and the diaphragm 70. Another tubular extension 96 projects outward from the annular sidewall 80 diametrically opposite the extension 84 and includes a passageway 98 extending radially from the central axis X and parallel to the longitudinal axis of the passageway section 86. The outer or free end of the passageway 98 is open and forms what can be referred to as the regulated suction port. The regulated suction port is configured to be connected to the suction adapter 24. The inner end of the passageway 98 terminates at the sidewall 80 and is open and in fluid communication with the lower chamber 94.

The lid or cover 68 is a generally cup-shaped member having a top wall 100 and a circular annular sidewall 102. The sidewall includes a pair of diametrically opposed notches 104 immediately adjacent the lower edge of the sidewall. The notches are configured to receive respective diametrically opposed projecting tabs 106 of the housing body to secure the lid or cover to the housing body and thus complete the housing assembly. The annular sidewall also includes an arcuate recess 108 in the lower edge of the sidewall 80 located midway between the notches 104 to receive the tubular extension 96 when the lid or cover 68 is secured to the housing body 78.

The piston 72 basically comprises a unitary body formed of a rigid plastic, such as ABS. The body includes a central hub 110 whose top end terminates in a circular flange 112. The top surface of the flange is planar but includes a circular bore 114 in the center thereof and extending into the hub. A plurality of reinforcing ribs (not shown) extends outward radially from the hub and serve to reinforce the flange and center the spring 76 about the central axis X. The bottom surface of the hub includes a recess 116 for receipt of the sealing disk 74. The sealing disk 74 is fixedly secured in the recess and serves as a valve member to engage the valve seat 92 in the lower chamber 94. The sealing disk 74 is formed of any suitable material, e.g., silicone rubber.

The diaphragm 70 is a rolling diaphragm formed of any resilient flexible material, e.g., Nitrile. The diaphragm includes a generally planar circular central portion 118 and a folded generally V-shaped or U-shaped edge portion 120 surrounding the central portion and terminating in a flanged generally planar thickened periphery 122. A small opening or hole 124 is in the center of the central portion 118. The central portion 118 is disposed on the planar top surface of the piston 72, with the thickened periphery 122 of the diaphragm disposed on an annular ledge 126 at the upper end of the sidewall 80.

With the lid or cover secured to the housing base the thickened periphery 122 of the diaphragm is tightly sandwiched between the ledge and the inner surface of the lid or cover. This arrangement divides the interior of the suction regulator into the heretofore identified lower chamber 94 and an upper chamber 128. The upper chamber is formed between the inner surface of the cover or lid and the upper surface of the diaphragm. The lower chamber 94 is formed between the inner surface of the sidewall 80 of the housing body and the undersurface of the portion of the diaphragm 70 located adjacent its periphery and a portion of the undersurface of the piston 72.

The cover or lid includes a small opening or vent 130 to the ambient atmosphere which will be referred to as the atmospheric reference port. The atmospheric reference port ensures that the upper chamber 128 will be at the pressure of the ambient atmosphere. The port 130 extends through the thickness of the cover and is in fluid communication with the interior of upper chamber to maintain that chamber at atmospheric pressure. Inasmuch as the atmospheric reference port is in the top surface of the cover, it is susceptible to being blocked by some object or even the finger of a user. To prevent such an occurrence the lid or cover includes a thickened portion 132 located adjacent the port 130 with an elongated shallow tripartite or T-shaped recess or channel 134 extending into the thickened portion. The outer or top end of the atmospheric reference port 130 is located at the bottom of the channel 134 at the intersection of the channel's various three sections and is in fluid communication with each of those sections. The outer end of each of the channel sections is open. Thus, if something should be on the top surface of the thickened portion 134 of the lid or cover disposed over the atmospheric reference port 130 air can still enter that port via any open end of the T-shaped channel 134.

The spring 76 is a helical compression spring formed of any suitable material, e.g., stainless steel. The spring is located within the lower chamber 94, with the upper end of the spring in engagement with the undersurface of the flanged portion 112 of the piston and surrounding a piston's central hub 110. The lower end of the spring is located within an annular recess 136 in the bottom wall of the housing body. The spring is under compression to bias the piston and diaphragm upward.

As mentioned above, the suction regulator 30 regulates the level of suction to a desired operating value, e.g., preferably approximately 200 mmHg, and provides the regulated suction to the suction assembly 22. To that end, the suction regulator 30 is configured to limit the amount of suction applied to the suction assembly to that desired value even if a level of suction greater than that predetermined value is applied to the suction regulator from the suction source (particularly if the suction source is at a much higher level, which will typically be the case if the suction source is the hospital's suction line). The predetermined or desired suction value (hereinafter referred to has the “regulator's set-point” or “regulated set-point value”) is fixed and is factory-established by the spring 76 and the dimensions of the housing body 78, the cover or lid 68, the piston 72, the sealing disk 74, and the stiffness of the diaphragm 70. In this regard the pressure within the upper chamber 128 will be equal to atmospheric pressure by virtue of the communication of that chamber with the ambient atmosphere via the atmospheric reference port 130. With suction applied, the pressure within the lower chamber 94 will be lower than the atmospheric pressure within the upper chamber 128. The differential pressure between the chambers 128 and 94 will force the diaphragm and the piston downward toward the valve seat 92. The spring 76, however, will impart a counter force on the piston and diaphragm that opposes the differential pressure force forcing the piston downward such that the level of suction appearing at the regulated suction port 98 is the desired operating value.

If the suction applied via line suction port 86 is greater that the predetermined value or level the piston and diaphragm will move such that the sealing disk 74 on the bottom of the piston's hub comes into engagement with the valve seat 92, thereby isolating the lower chamber 94 from the suction appearing on the line suction port. This action thereby limits the level of suction in lower chamber to the predetermined level (operating value). If, however, the suction applied via line suction port is less than the predetermined operating level the piston and diaphragm will only move part of the way downward. As such the level of suction applied to the line suction port will equal that in the regulated suction port and that applied to the suction assembly 22.

The suction regulator 30 is configured to prevent the sealing disk 74 from becoming stuck for an extended period on the valve seat 92 in the event of what will be referred to hereinafter as an “over-travel situation”. In this regard, it has been determined that if the suction regulator is operated in a manner such that a high level of suction is applied very rapidly, the piston may experience an over-travel wherein it moves downward very quickly such that the sealing disk becomes stuck on the valve seat. Under this condition the suction assembly 22 would have a higher level of vacuum (suction) than the regulator 30 was set to provide, e.g., approximately 200 mmHg. The regulator could stay in that state for an extended/indefinite period. To prevent such an occurrence, the suction regulator 30 includes two “bleed holes”. One bleed hole, hereinafter referred to as the first bleed hole, is the heretofore identified small hole 124 located in the center of the diaphragm on the central axis X. The second bleed hole (not shown) is in a radially extending recess (not shown) in a cylindrical cavity (not shown) in the piston 72 contiguous with the planar top surface of the piston's flanged portion 112. The radially extending recess is in fluid communication with the cylindrical cavity. The second bleed hole is located at the bottom of the radially extending recess and extends through the flanged portion of the piston to the underside of the flanged portion. The first bleed hole is in fluid communication with the cylindrical cavity in the piston. Thus, the second bleed hole (which is in the piston) will be in fluid communication with the first bleed hole. Since the first bleed hole is in communication with the upper chamber 128, that chamber will be in fluid communication with the lower chamber 94 via the communicating bleed holes. Hence, if the sealing disk should become stuck on the valve seat, air which enters the upper chamber via the atmospheric reference port can then pass through the bleed hole in the piston, from whence it will enter into the lower chamber 94. The ingress of air into the lower chamber will decrease the vacuum within that chamber, thus enabling the spring 76 to move the piston upward so that the sealing disk 74 is off the valve seat 92.

As mentioned earlier, the system shown in FIG. 1 includes two embodiments of a locking clip, namely, clip 200 and clip 300. The clip 200 is a low-profile device that basically comprises a generally planar disk of circular profile and includes a radially extending slot 138 from the center of the disk to its circular periphery 140. The clip 200 is formed of any suitably rigid plastic, e.g., nylon, and has a diameter of 1.5 inch and a thickness of 0.25 inch. The slot 138 is approximately 0.9 inch wide to provide a 0.035 inch interference fit with the outside diameter of the suction tube 34 when the clip is used on the suction tube to fix the target tissue/organ which is releasably secured to the suction head at its desired position within the body of the patient, as will be described later. Moreover, due to its relative thickness, the wall of the suction tube 34 prevents complete collapse of the flow path through it when the clip 200 is applied onto the suction tube during use.

The clip 300 is more preferred embodiment of a locking clip constructed in accordance with this invention. Like the clip 200 the clip 300 basically comprises an integral structure formed of a hard flexible polymer, e.g., nylon, but is shaped differently, i.e., is in the general shape of a pliers. In particular, the clip 300 consists of a one-piece or integral unit having a pair of jaw sections 302, a pair of handle sections 304, and an elongated channel or slot 306 located at the interface between the jaw sections and handle sections. The slot is open at its front or distal end. The proximal or rear end of the slot is in the form of an arcuate bridge section 308. The inner surfaces of jaw sections confront each other to form an entryway 310 to the elongated slot 306. In its normal or quiescent state, the clip 300 is as shown in FIG. 1. In that state the width of the slot is approximately 0.09 inch wide at the open/front end, and 0.10 in wide on the rear end. That configuration effectively secures the clip to the suction tube as the tube approaches the entryway and avoid the tube preferentially positioning itself directly near the jaws where it would be more likely to slip out of the clip. The width of the entryway to the slot is approximately 0.03 inch, whereupon the open distal end of the slot is slightly smaller than the width of the slot. The width of the slot in its normal or quiescent state provides a 0.03 inch interference fit with the outside diameter of the suction tube 34 when the clip is used on the suction tube to fix the target tissue/organ which is releasably secured to the suction head at its desired position within the body of the patient, as will be described later. The bridge section 308 is configured to flex, e.g., to act as a hinge, to enable a portion of the suction tube 34 to be introduced into the slot 306 via the entryway 310. To that end, the handle sections 304 are configured to be squeezed together by a user, which action causes the bridge section 208 to flex thereby increasing the width of the entryway 210 to the slot 206, so that the clip can be placed onto the suction tube to releasably lock the clip in place.

In accordance with one exemplary embodiment of the system 20 of FIG. 1, the suction adapter 26 is a conventional push-connect adapter, e.g., like that sold by Grainger, Inc., under the model designation/part number 36W672, “Brass, Push-to-Connect x Push-to-Connect, For ⅛ in ×¼ in Tube OD”. As best seen in FIGS. 15-17 the adapter 26 basically comprises a slidable throat member 142, a retention ring 144, a gasket 146, and a tubular body 148. The tubular body 148 is formed of a hard plastic, e.g., nylon, and includes a large diameter bore 150 at one end, a small diameter bore 152 at the opposite end, and an intermediate bore 154 located between the large diameter bore and the small diameter bore. The slidable throat member 142 is a tubular member formed of a hard plastic, e.g., nylon, having a central bore 156 and a surrounding flanged end 158. The gasket is a ring-like member surrounding a central opening. The gasket is formed of a resilient material. The slidable throat member, the retention ring 144, and the gasket 146 are in the large diameter bore 152, with the gasket being in engagement with the portion of the tubular body surrounding the intermediate diameter bore 154.

The central bore 156 of the slidable throat member is configured to receive the free end 34B of the suction tube 34 to connect the suction tube to it. The retention ring is formed of metal, e.g., brass, and includes plural teeth 160 configured to engage the outer surface of the suction tube contiguous with the free end thereof when the free end of the suction tube is inserted into the central bore 156. In particular, the free end of the suction tube, which is semi-rigid, is inserted into the throat member, past the teeth of the retention ring and through the central opening in the gasket. The teeth 160 are arranged to form a circle, with a diameter smaller than the outside diameter of the suction tube contiguous with its free end 34B. The teeth are angled away from the throated end of the adapter 26, so that they act like barbs. Once the suction tube contiguous with the free end thereof is pushed past the teeth and through the central passageway in the gasket, the teeth dig into the suction tube and retain it within the adapter 26, with the gasket forming an interference fit with the suction tube thereby creating a fluid-tight seal therebetween.

The end of the tubular body 148 surrounding the small diameter bore 152 of the suction adapter 26 has an outside diameter sufficient to tightly receive the open end of the flexible tube 12 to connect the adapter 26 to the conventional suction regulator (not shown) as will be described later.

When one wants to disconnect the suction adapter 26 from the suction tube 34, all that is required is to push the flange 158 of the throat member into the bore 150 against the teeth. This applies a force to the teeth causing them to bend out and away from the suction tube to allow the suction tube to be withdrawn out of the adapter.

The suction adapter 24 is best shown in FIGS. 9-11 and basically comprises a unitary tubular body 162 formed of a medium-hard plastic, e.g., polyurethane, which includes a large diameter bore 164 at one end, a small diameter bore 166 at the opposite end, and an intermediate bore 168 located between the large diameter bore and the small diameter bore. The large diameter bore 164 is configured to tightly receive the regulated suction port extension 96 of the suction regulator 30. To facilitate the introduction of the suction port extension 96 into the bore 164, the open end 170 of the bore is chamfered. The small diameter bore 166 of the suction adapter 24 is configured to tightly receive the free end of the suction tube 34. To facilitate the introduction of the free end of the suction tube 34 into the bore 166, the open end 172 of the bore is chamfered. The intermediate bore 168 is provided to maintain a consistent length of the suction tube 34 between the suction adapter 24 and suction head 32. In accordance with one exemplary embodiment of the adapter 24 the inside diameter of its intermediate bore is 0.10 inch. The relative softness of the adapter 24 maintains an airtight seal with both the relatively hard suction tube 34 and rigid tubular extension 96.

One exemplary preferred method of use of the system 20 will now be described with reference to FIGS. 18A-18L. A conventional trocar 6 is introduced through the abdominal wall 8 so that it extends into the abdominal cavity adjacent the location of the target tissue/organ, e.g., the liver 2, to be positioned. Once the trocar is in place extending through the abdominal wall into the abdominal cavity such as shown in FIG. 18A, the suction assembly 22 (i.e., the suction head 32, the suction tube 34 and the retraction filament 36) is ready for introduction as a unit into the abdominal cavity. To that end, the suction head 32 is folded closed lengthwise to collapse it. Moreover, the nipple 50 of the suction head is folded down into the surrounding annular recess 52 and the contiguous section of suction tubing 34 is folded or otherwise bent into one of the linear recesses 54 and 56 in the top wall portion of the suction head. This action results in the cross-sectional area of the suction head and the contiguous section of suction tubing being reduced considerably to facilitate the introduction and passage of the suction head through the trocar as shown in FIG. 18B.

One or more laparoscopic graspers (not shown) may be used to push and grab the suction head through the remaining length of the trocar and into the abdominal cavity. The suction tube 34 and connected retraction filament 36 is fed/pulled into the abdominal cavity following the suction head as shown in FIG. 18C. Once the suction assembly is within the abdominal cavity an optimal retraction point 18 is identified above the target tissue/organ within the abdominal cavity, whereupon a conventional suture passer 14 (e.g., a Carter Thomason device) is inserted into the abdominal cavity at the identified point on the abdominal wall as shown in FIG. 18D. Once inside the abdomen, the jaws 14A of the suture passer 14 are opened and positioned on opposite sides of the free end 36A of the retraction filament 36. The jaws are then closed to grasp the free end of the retraction filament. The suture passer 14 can then be retracted as shown FIG. 18E to pull the retraction filament and several inches of the suction tube out of the patient as shown in FIG. 18F, whereupon the extending externalized portion of the retraction filament 36 can be grasped and pulled further outward as shown in FIG. 18G. Continued retraction of the retraction filament brings the connected heat-formed tip 62 of the suction tube into the opening formed at the identified point 18 by the suture passer as shown in FIG. 18H. Continued retraction of the retraction filament brings the heat-formed conical tip 62 of the suction tube out of the patient's body as shown in FIG. 18I, whereupon the externalized conical tip 62 can be cut off by a scissors 16 (or some other cutting instrument) as shown in FIG. 18I. That action results in the removal of the retraction filament from the suction assembly 22 and forms a free end 34B of the suction tube as shown in FIG. 18J. The free end 34B is open and in fluid communication with the central passageway 34A in the suction tube.

The suction head 32 can then be moved by means of a conventional grasper (not shown) so that it is juxtaposed over the target tissue/organ, e.g., the liver 2, as shown in FIG. 18J and from there into very close proximity or actual engagement with the target tissue/organ as shown in FIG. 18K. Once the suction head is in position the open free end 34B of the suction tube can then be connected to a source of regulated suction to provide regulated suction at the desired regulated value to the suction tube. That regulated suction is transmitted through the suction tube 34 and the nipple 50 to the foam insert 40 in the suction head and thus into the operating field 4. The suction head can then be placed onto the target tissue/organ 2 so that a seal is formed between the flanged peripheral edge 46 of the suction head and the target tissue/organ. The foam inserts 40 inside the cavity of the suction head prevents the suction head from sliding along the surface of the target tissue/organ and prevents the target tissue/organ from completely filling the suction head and greatly reducing the lifting force.

In accordance with one aspect of the system and method of this invention, the regulated suction can be provided to the suction head 32 in either of two ways. One way is as shown in FIG. 2A, where the free end 34B of the suction tube is connected to the suction adapter 24 and its associated suction regulator 30. The line suction port formed by the open end of passageway 86 of the extension 84 is connected via a section of conventional tubing 10 to a wall/line suction source (not shown). Accordingly, the high suction level of the wall/line suction source is reduced by the suction regulator 30 to the desired regulated value.

The other way that regulated suction can be provided to the suction head 32 is shown FIG. 2B, whereupon the open free end 34B of the suction tube is connected within the large diameter bore 150 at one end of the suction adapter 26 as described earlier. The portion of the body 148 of the suction adapter 26 surrounding the small diameter bore thereof is inserted into the open end of a section of conventional tubing 12 as also described earlier. That section of tubing 12 is in turn connected to any conventional regulated suction source (not shown) which is configured to provide regulated suction at the desired value to the suction assembly 22.

In either case, once the suction head 32 is in position the externalized suction tube 34 can be pulled out of the patient until the target tissue/organ is brought to the desired retracted position, allowing for unobstructed visualization of the relevant anatomy. The suction tube is then releasably secured in place externally using the locking clip 200 or the more preferred locking clip 300.

With respect to the locking clip 200 the releasable securement of the system with the target tissue/organ in its desired retracted position is achieved by inserting a portion of the suction tube within the slot 138 of the clip 200 and sliding the clip along the suction tube 34 just above the patient's abdomen, such that the clip rests on the exterior of the abdomen. This prevents the suction head and target tissue/organ from slipping back into the operating field. The amount of provided retraction (or the distance the target tissue/organ is moved from its natural position) can be adjusted by removing the locking clip 200, lifting or lowering the target tissue/organ via the externalized suction tube, and then re-clipping the suction tube at the desired height by use of the clip 200. This action stabilizes and holds the target tissue/organ at a desired position.

The releasable securement of the system with the target tissue/organ in its desired retracted position is achieved by use of the preferred locking clip 300 in the following manner. The clip 300 is positioned so that a portion of the suction tube which extends out of the abdomen of the patient is located between the jaw sections 304 of the clip and then moving the clip, whereupon that portion of the suction tube passes through the entryway 310 to the slot 306. The clip 300 can then be moved downward with respect to the suction tube so that the undersurface of the clip rests on the exterior of the abdomen of the patient. When that has occurred, the handle sections 304 can be released, whereupon the natural bias of the material making up the bridge section or hinge 308 causes the clip 300 to reassume its normal or quiescent state, thereby squeezing the portion of the suction tube within the slot 306, so that the clip is releasably locked in place by the frictional engagement between the surfaces of the slot and the surface of the suction tube. This action prevents the suction head and target tissue/organ from slipping back into the operating field. The amount of provided retraction (or the distance the target tissue/organ is moved from its natural position) can be adjusted by re-squeezing the handle sections 304 to release the grip of the clip on the suction tube and then lifting or lowering the target tissue/organ via the externalized suction tube, and re-clipping the suction tube at the desired height by releasing the handle sections 304 of the clip 300 when it is in the desired position. That action stabilizes and holds the target tissue/organ at the desired position.

As should be appreciated by those skilled in the art the benefit of the clip 300 over the clip 200 is that the clip 300 can be placed and readjusted with minimal strain on the user.

When positioning of the target tissue/organ is no longer needed, the target tissue/organ can be lowered back down and released from the suction assembly by breaking the suction seal. This can be accomplished by lifting a portion of the peripheral edge of the suction head away from the target tissue/organ or by deactivating the source of suction. That action frees the suction assembly from the target tissue/organ. Once that has been accomplished the suction assembly can then be removed from the patient with a placed trocar by gripping the suction head 32 longitudinally with a laparoscopic grasper and removing the trocar and grasper simultaneously. If desired, a tissue specimen may be taken of the target tissue/organ within the abdominal cavity and then bagged for removal. That bagging and removal action may be accomplished by capturing the tissue specimen within a laparoscopically deployable specimen bag and removing it from the patient via the opening created by a trocar. The system remaining within the patient may then also be removed through this opening.

It must be noted at this point that while the system 20 of the subject invention has been described for retracting the liver of a patient, the system and its method of use is not limited to a particular organ or tissue. Systems constructed in accordance with this invention and their methods of use can be for elevating, retracting, or otherwise positioning other organs, tissues, or other anatomic structures within the body of a patient during a laparoscopic or robotic procedure. In fact, the subject system and its methods of use can be implemented in open surgical procedures where the surgical access incision is not extensive. Moreover, the exemplary components making up the system 20 may be changed from those specifically described. For example, other conventional suction adapters can be used in lieu of the adapter 26 interposed between a conventional suction regulator and the suction assembly like shown in FIG. 2B. In fact, various changes can be made to the components of the system and its methods of use, other than those specifically described or disclosed above. For example, the materials making up the components of the suction assembly, can be different than those disclosed above. Moreover, the dimensions of the suction head and the suction tube can be different than those disclosed above of a system for positioning the liver of an adult through a standard 12 mm trocar.

In any case, as should be appreciated by those skilled in the art, the systems and methods of this invention provide clinical advantages over the prior art systems/methods. For example, the subject invention is easily adjustable without the need to maneuver external mounts or reposition internally fixated components, e.g., internal hooks, placed sutures, etc. Moreover, the use of the subject system is compatible with (fits) into traditional surgical workflows so that there is a low learning curve, and no specialty tooling is required for use. Moreover, the low external profile of the suction assembly requires no external fixtures that occupy space above the patient and interfere with robotic arms. Furthermore, the subject invention does not require an extra patient port so that it reduces the chance of infection and operative time. Further still, the suction assembly of the system of this invention can be left active and unengaged in the surgical space without disrupting pneumoperitoneum due to extremely low flow rate, e.g., 6 L/min. Lastly, the use of an integrated suction regulator, like that shown in the FIG. 2A, can prevent tissue damage due to user error or defective hospital equipment.

Without further elaboration the foregoing will so fully illustrate our invention that others may, by applying current or future knowledge, adopt the same for use under various conditions of service.

We claim:

1. A system for positioning an anatomic structure at a desired position within the body of a patient, said system comprising:
a suction assembly comprising a collapsible suction head, a flexible suction tube, and a retraction filament, said collapsible suction head being a hollow member having an internal cavity in which a foam insert is located and a peripheral edge extending about the periphery of said cavity, said collapsible suction head including a suction port, said flexible suction tube having a first end including a first opening coupled to and in fluid communication with said suction port, a second end including a second opening, and a passageway extending between said first and second openings, said retraction filament being an elongated flexible member having a first end and a second end,—said first end being anchored to said flexible suction tube adjacent said second opening, said suction assembly being configured to be introduced through a trocar or other port into the body of the patient, whereupon said collapsible suction head is located within the body of the patient, with a portion of said flexible suction tube and a portion of said retraction filament extending out of the body of the patient through the trocar or other port;
a suction adapter configured to be connected said passageway of said flexible suction tube and to a source of suction; and
a clip configured to be releasably secured to a portion of said flexible suction tube located outside the body of the patient to hold said suction assembly and the anatomic structure at a desired position within the body of the patient.

2. The system of claim 1, wherein said retraction filament is configured to be retracted to pull said second end of said flexible suction tube outside of the body of the patient, whereupon a portion of said flexible suction tube contiguous with said second end of said flexible suction tube can be removed to form a free end of said flexible suction tube and disconnecting said retraction filament from said flexible suction tube.

3. The system of claim 2, wherein said suction assembly is configured so that said peripheral edge of said collapsible hollow head can be brought into engagement with the anatomic structure within the body of the patient.

4. The system of claim 2, wherein said suction adapter is configured to be connected to said passageway of said flexible suction at said free end of said flexible suction tube.

5. The system of claim 4, wherein when said suction adapter is connected to said passageway suction from said source of suction is applied to the interior of said internal cavity and through said foam insert to cause the anatomic structure to be releasably secured by suction to said suction head.

6. The system of claim 1, wherein said first end of said retraction filament is anchored to said flexible suction tube within said passageway adjacent said second opening.

7. The system of claim 1, wherein said flexible suction tube has an outside diameter of a first dimension, and wherein said clip comprises a slot having a width which is less than said first dimension.

8. The system of claim 1, additionally comprising a suction regulator configured to be connected between said suction adapter and the source of suction for regulating the level of suction provided by the source of suction.

9. The system of claim 8, wherein said suction regulator comprises a first chamber, a second chamber, a movable diaphragm and a biasing member, said first chamber being configured to have suction applied thereto from said source of suction, said second chamber being at atmospheric pressure, said movable diaphragm being disposed within said suction regulator separating said first chamber from said second chamber, whereupon a differential pressure exists between said first and second chambers, said differential pressure imparting a differential pressure force on said movable diaphragm, said biasing member being configured to impart a counter force on said movable diaphragm that opposes said differential pressure force.

10. The system of claim 9, wherein said first chamber comprises a valve seat and a movable sealing member coupled to said movable diaphragm, said valve seat surrounding an opening for fluid within said first chamber to flow therethrough, said sealing member blocking said opening when said differential pressure force exceeds said counter force imparted by said biasing member.

11. The system of claim 1, wherein said suction adapter is configured to be connected to a section of flexible tubing connected to the source of suction and wherein the source of suction is regulated.

12. The system of claim 1, wherein said foam insert comprises an open cell foam.

13. The system of claim 1, wherein said collapsible suction head comprises a top portion and a downwardly projecting peripheral skirt, said downwardly projecting peripheral skirt terminating in said peripheral edge, said suction port being in said top portion of said collapsible suction head.

14. The system of claim 13, wherein said top portion of said collapsible suction head includes a recess configured for receipt of a portion of said flexible suction tube.

15. The system of claim 1, wherein said flexible suction tube includes a stretchable coil section located closely adjacent said collapsible suction head.

16. A method of positioning an anatomic structure at a desired position within the body of a patient, said method comprising:
providing a suction assembly comprising a collapsible suction head, a flexible suction tube, and a retraction filament, said collapsible suction head being a hollow member having an internal cavity in which a foam insert is located and a peripheral edge extending about the periphery of said cavity, said collapsible suction head including a suction port, said flexible suction tube having a first end including a first opening in fluid communication with said suction port, a second end including a second opening, and a passageway extending between said first and second openings, said retraction filament having a first end and a second end, said first end of said retraction filament being anchored to said flexible suction tube adjacent said second opening;
introducing said suction assembly through a trocar or other port into the body of the patient, whereupon said collapsible suction head is located within the body of the patient, with a portion of said flexible suction tube and said retraction filament extending out of the body of the patient through the trocar or other port;
retracting said retraction filament through said trocar or other port from outside the body of the patient to pull said proximal end of said flexible suction tube outside of the body of the patient through an exit port in the body of the patient;
removing a section of said flexible suction tube contiguous with said second end of said flexible suction tube outside the body of the patient to form a free end of said flexible suction tube and disconnecting said retraction filament from said flexible suction tube;
connecting a suction adapter to said free end of said flexible suction tube and to a source of suction;
bringing said peripheral edge of said collapsible suction head is in engagement with the anatomic structure within the body of the patient; and
applying suction at a predetermined value to said interior of said internal cavity and through said foam insert to cause the anatomic structure to be releasably secured by suction to said collapsible suction head.

17. The method of claim 16, additionally comprising:
positioning said suction assembly with the anatomic structure releasably secured thereto at a desired location within the body of the patient.

18. The method of claim 17, additionally comprising:
retracting said flexible suction tube to remove any slack in said flexible suction tube between said collapsible head and an exterior surface of the body of the patient at said exit port, and releasably securing a clip to a portion of said flexible suction to outside the body of the patient contiguous with said exit port.

19. The method of claim 16, wherein the anatomic structure is an organ located within the abdomen of the patient.

20. The method of claim 16, wherein said predetermined value is within the range of approximately 150 to 300 mmHg.
